(19) United States
(12) Patent Application Publication
(10) Pub. No.: US 2016/0030502 A1
(43) Pub. Date: Feb. 4, 2016

(54) Eye Health Supplement

(71) Applicants: Adam C. Saucedo, Sandy, UT (US); Balamurali K. Ambati, Salt Lake City, UT (US)

(72) Inventors: Adam C. Saucedo, Sandy, UT (US); Balamurali K. Ambati, Salt Lake City, UT (US)

(21) Appl. No.: 14/817,973

(22) Filed: Aug. 4, 2015

Related U.S. Application Data
(60) Provisional application No. 62/032,796, filed on Aug. 4, 2014.

(51) Int. Cl. A61K 38/06; A23L 1/30; A61K 36/185; A61K 36/45; A61K 36/48; A61K 31/12; A61K 31/122; A23L 1/29; A61K 36/88

(52) U.S. Cl. CPC A61K 38/063; A23L 1/296; A23L 1/3002; A61K 36/185; A61K 36/88; A61K 36/48; A61K 31/12; A61K 31/122; A61K 36/45; A23V 2002/00

(57) ABSTRACT

An eye health supplement administered to a person in order to prevent and treat eye-related conditions or diseases. The eye health supplement focuses on treating conditions and diseases, such as diabetic retinopathy, macular degeneration, hypertensive retinopathy, keratoconjunctivitis sicca, and more, and the underlying symptoms of eye-related conditions and diseases through the inclusion of a quantity of birch leaf powder, a quantity of pomegranate, a quantity of garlic bulb powder, a quantity of fenugreek powder, a quantity of curcumin, a quantity of asthaxanthin, a quantity of bilberry extract powder, and a quantity of glutathione powder. The quantity of birch leaf powder, the quantity of pomegranate, the quantity of garlic bulb powder, the quantity of fenugreek powder, the quantity of curcumin, the quantity of asthaxanthin, the quantity of bilberry extract powder, and the quantity of glutathione powder are heterogeneously mixed into a supplement mixture for oral or transdermal administration.

The current application claims a priority to the U.S. Provisional Patent application Ser. No. 62/032,796 filed on Aug. 4, 2014.

### FIELD OF THE INVENTION

The present invention relates generally to healthcare supplements and related nutritional products. More specifically, the present invention is an eye health supplement that is beneficial in the prevention and treatment of a number of eye-related diseases and other conditions.

### BACKGROUND OF THE INVENTION

Proper eye healthcare is a critical component in maintaining one's overall health. Improper or negligent care of the eyes can contribute to a multitude of eye-related diseases and other conditions as well as result in impaired vision or effect daily activities. Eye-related diseases and conditions may be minor and result in relatively insignificant symptoms such as slight inflammation or irritation. However, more serious diseases and conditions may result in significant eye damage and decreased quality of vision or loss of vision altogether. Diabetic retinopathy is a complication of diabetes that can eventually lead to blindness. The disease is characterized by the formation of abnormal blood vessels at the back of the eyes that burst and bleed, blurring vision. Macular degeneration is a condition that results in the loss of vision due to damage to the retina. The condition causes distorted vision in the macula region of the retina while peripheral vision is relatively unaffected. Hypertensive retinopathy is linked to high blood pressure (hypertension) and is characterized by damage to the retina and retinal circulation leading to decreased quality of vision. Keratoconjunctivitis sicca (dry eye syndrome) is a condition that is caused by decreased tear production or increased tear film evaporation. The condition is characterized by significant discomfort in the eyes such as dryness, burning, itchiness, stinginess, redness, and pain. A very important aspect of eye healthcare is the intake of appropriate nutrition required to achieve and maintain proper eye health.

The present invention seeks to address the aforementioned issues related to eye health care by supplying a supplement with the focus of preventing and treating diabetic retinopathy, macular degeneration, hypertensive retinopathy, and keratoconjunctivitis sicca, as well as other eye related conditions and diseases. The present invention is designed to relieve symptoms and underlying causes of mentioned or otherwise eye related conditions.

### DETAIL DESCRIPTIONS OF THE INVENTION

All illustrations of the drawings are for the purpose of describing selected versions of the present invention and are not intended to limit the scope of the present invention.

Eye-related diseases and conditions have a negative effect on a person vision and can lead to impaired vision or blindness. The present invention is an eye health supplement focusing treating and alleviating diabetic retinopathy, macular degeneration, hypertensive retinopathy, and keratoconjunctivitis sicca; however, the present invention has applications for treating and alleviating other eye-related diseases and conditions. Diabetic retinopathy is a complication of diabetes which damages blood vessels of the retina of an eye resulting in severe loss of vision or blindness. Macular degeneration is a disease caused by the deterioration of the central portion of the retina leading to severe loss of vision. Hypertensive retinopathy refers to damage of the retinal vascular system from high blood pressure leading to decreased quality of vision. Keratoconjunctivitis sicca is a condition due to decreased tear production or increased tear film evaporation causing significant discomfort such as dryness, burning, itching, redness and pain in the eyes. The present invention focuses on treating the symptoms and underlying causes such eye-related diseases.

The present invention comprises a quantity of birch leaf powder, a quantity of pomegranate, a quantity of garlic bulb powder, a quantity of fenugreek powder, a quantity of curcumin, a quantity of asthaxanthin, a quantity of bilberry extract powder, and a quantity of glutathione powder in order to treat symptoms of eye-related diseases and conditions by providing a plurality of medicinal effects. The quantity of birch leaf powder, the quantity of pomegranate, the quantity of garlic bulb powder, the quantity of fenugreek powder, the quantity of curcumin, the quantity of asthaxanthin, the quantity of bilberry extract powder, and the quantity of glutathione powder are heterogeneously mixed into a supplement mixture such that the supplement mixture is easily administered. The supplement mixture is administered orally, through use of capsules, powders, compacted tablets, or a drinkable liquid; however, the supplement mixture can also be administered through transdermal methods such as patches.

TABLE 1

| | Percent by Weight of |
|---|---|
| Compound | the Supplement Mixture |
| Birch Leaf Powder | 3.2% |
| Pomegranate | 6.5% |
| Garlic Bulb Powder | 6.5% |
| Fenugreek Powder | 32% |
| Curcumin | 16% |
| Asthaxanthin | 0.3% |
| Bilberry Extract Powder | 19.4% |
| Glutathione Powder | 16% |

In accordance to Table 1, the quantity of birch leaf powder is approximately 3.2% by weight (wt) of the supplement mixture to provide sufficient salicylates, the salts and esters of salicylic acid, which impart ocular anti-inflammatory properties to the supplement mixture. Anti-inflammatory properties are beneficial in alleviating burning, itching and pain associated with keratoconjunctivitis sicca and other eye-related conditions and diseases. The quantity of bilberry extract powder is approximately 19.4% wt of the supplement mixture. The quantity of bilberry extract powder provides ocular anti-inflammatory properties to the supplement mixture, as well as, inhibiting or reversing macular degeneration and treating retinopathy due to the presence of anthocyanosides that protect the retina.

The quantity of fenugreek powder has been shown to slow the absorption of sugar through ingestion and stimulate insulin and, therefore, is effective in regulating a user's blood sugar levels. Properly regulating blood sugar levels is effective in preventing and treating diabetic retinopathy. The quantity of fenugreek powder is approximately 32% wt of the supplement mixture.

The quantity of garlic powder is approximately 6.5% wt of the supplement mixture and provides the supplement mixture with the compound allicin. Allicin, an organosulfur compound derived from garlic exhibits antibacterial, anti-inflammatory, antithrombotic, antioxidant, and antifungal properties while simultaneously decreasing blood pressure. Because hypertensive retinopathy is correlated with high blood pressure, the quantity of garlic powder helps to prevent and treat hypertensive retinopathy by reducing the strain imposed on the retinal blood vessels.

The quantity of curcumin, the quantity of pomegranate, the quantity of asthaxanthin, and the quantity of glutathione powder provide beneficial antioxidants to the user through administration of the present invention as well as other beneficial medical properties. Antioxidants aid in reducing oxidative stress of the eyes caused by daily exposure to sunlight, high metabolic activities, and oxygen tension as well as combating free radicals that damage blood cells and prevent oxidation of molecules which cause cells to function poorly or die, slowing the progression of macular degeneration. The quantity of curcumin is approximately 16% wt of the supplement mixture and provides beneficial medicinal compounds, including diarylheptanoid. Diarylheptanoid is derived from the spice turmeric and exhibits anticancer effects in addition to the antioxidant. The quantity of curcumin has displayed properties which decrease blood sugar and blood pressure. The quantity of pomegranate is approximately 6.5% wt of the supplement mixture. The quantity of pomegranate possess punicalagin isomers and a high phenolic content resulting in a high antioxidant potential. Pomegranate is a widely cultivated and consumed fruit and known to possess a large amount of antioxidants. The quantity of asthaxanthin is approximately 0.3% wt of the supplement mixture. Asthaxanthin is a carotenoid found in micro algae, yeast, salmon, trout, hill, shrimp, and crustaceans. Asthaxanthin has shown to have potent antioxidant effects on cardiovascular, immune, inflammatory, and neurodegenerative diseases. The quantity of glutathione powder is approximately 16% wt of the supplement mixture. Glutathione is a tripeptide found in a wide variety of plants and animals which serves as an antioxidant for combating free radicals and reactive oxygen compounds as well as maintaining other antioxidants such as vitamin C and vitamin E in reduced and active forms. Antioxidants inhibit the oxidation of molecules that can lead to the formation of free radicals that may react with cellular components such as DNA or cell membranes.

Although the invention has been explained in relation to its preferred embodiment, it is to be understood that many other possible modifications and variations can be made without departing from the spirit and scope of the invention as hereinafter claimed.

What is claimed is:

1. An eye health supplement comprises:
a quantity of birch leaf powder;
a quantity of pomegranate;
a quantity of garlic bulb powder;
a quantity of fenugreek powder;
a quantity of curcumin;
a quantity of asthaxanthin;
a quantity of bilberry extract powder;
a quantity of glutathione powder; and
the quantity of birch leaf powder, the quantity of pomegranate, the quantity of garlic bulb powder, the quantity of fenugreek powder, the quantity of curcumin, the quantity of asthaxanthin, the quantity of bilberry extract powder, and the quantity of glutathione powder being heterogeneously mixed into a supplement mixture.

2. The eye health supplement as claimed in claim 1 comprises:
the quantity of birch leaf powder being approximately 3.2% by weight (wt) of the supplement mixture.

3. The eye health supplement as claimed in claim 1 comprises:
the quantity of pomegranate being approximately 6.5% wt of the supplement mixture.

4. The eye health supplement as claimed in claim 1 comprises:
the quantity of garlic bulb powder being approximately 6.5% wt of the supplement mixture.

5. The eye health supplement as claimed in claim 1 comprises:
the quantity of fenugreek powder being approximately 32% wt of the supplement mixture.

6. The eye health supplement as claimed in claim 1 comprises:
the quantity of quantity of curcumin being approximately 16% by weight of the supplement mixture.

7. The eye health supplement as claimed in claim 1 comprises:
the quantity of asthaxanthin being approximately 0.3% by weight of the supplement mixture.

8. The eye health supplement as claimed in claim 1 comprises:
the quantity of bilberry extract powder being approximately 19.4% by weight of the supplement mixture.

9. The eye health supplement as claimed in claim 1 comprises:
the quantity of glutathione powder being approximately 16% by weight of the supplement mixture.
